(19) United States
(12) Patent Application Publication
(10) Pub. No.: US 2022/0369962 A1
(43) Pub. Date: Nov. 24, 2022

(54) A BLOOD GLUCOSE MONITORING DEVICE AND SYSTEM

(71) Applicant: JIAXING SUMMED MEDTECH CO., LTD., Jiaxing, Zhejiang (CN)

(72) Inventors: Jiansen WENG, Jiaxing, Zhejiang (CN); Dehui LI, Jiaxing, Zhejiang (CN)

(21) Appl. No.: 17/765,339

(22) PCT Filed: Dec. 13, 2019
(86) PCT No.: PCT/CN2019/125232

§ 371 Date: Mar. 30, 2022

(30) Foreign Application Priority Data
Oct. 29, 2019 (CN) 201911039959.5
Oct. 29, 2019 (CN) 201921849294.X

(51) Int. Cl. A61B 5/145; A61B 5/151; A61B 5/15

(52) U.S. Cl. CPC A61B 5/14532; A61B 5/151; A61B 5/15087; A61B 5/150305; A61B 2560/0431

(57) ABSTRACT

A blood glucose detection device and system are portable blood glucose detection devices for patients. The blood glucose detection device includes a plurality of lancing units, which collect patient blood and generate current. The test unit can detect the current and transmit the current intensity data to the external device, thus making the glucose detection device portable. Besides, there is no need to change test strips every time for a blood glucose test as the current intensity data can be transmitted externally for management, which improves the patient's experience and makes it convenient for patients to perform blood glucose tests when they go out.

### FIELD OF DISCLOSURE

The application relates to the field of intelligent healthcare, in particular to a blood glucose detection device and system.

### BACKGROUND OF THE INVENTION

The current equipment that tests blood glucose is mainly a blood glucose detection device, but it is inconvenient to carry when the patient goes out, and is also relatively cumbersome during use as a new test strip needs to be replaced every time a test is performed. Besides, it is difficult to transmit data in the blood glucose detection device externally for management. Therefore, it is necessary to provide a portable blood glucose detection device, making it convenient for patients to use when they go out.

### SUMMARY OF INVENTION

One object of the application is to provide a kind of blood glucose detection device and system, for avoiding the problem that existing blood glucose detection device is inconvenient to carry and blood glucose test strips needs to be replaced when using.

In order to achieve the above object, the application provides a blood glucose detection device, wherein: the device comprises a portable card-type base, at least one lancing unit and a test unit. At least one lancing unit is arranged on the edge of the portable card-type base. The lancing unit includes a groove provided on the portable card-type base, the groove is coated with glucose oxidase or glucose dehydrogenase and the lancing unit is used to collect blood and generate electric current. The test unit is arranged on the portable card-type base and electrically connected with the lancing unit, and is used for detecting the intensity of the generated current and communicating with the outside.

Furthermore, there are a plurality of lancing units. The amount is between 2 and 20.

Furthermore, these lancing units are provided with intervals between two, and are provided along the edge of the portable card-type base.

Furthermore, these lancing units are also covered with a protective film, which is equipped with a siphon groove. And the opening of the siphon groove is located at the edge of the portable card-type base and communicated with the outside.

The test unit further includes a communication module and a current detection module, and the communication module is electrically connected to the current detection module.

Furthermore, the communication module is a near field communication chip.

The device further includes a temperature detection unit, which is equipped on the portable card-type base, and is electrically connected with the communication module.

According to another aspect of the application, the application provides a blood glucose detection device, wherein: the blood glucose detection device comprises a portable housing with cavity, lancet, at least one lancing unit and a test unit. At least one lancing unit is arranged on the cavity opening side of the portable housing. The lancing unit includes a groove provided on the portable housing, the groove is coated with glucose oxidase or glucose dehydrogenase and the lancing unit is used to collect blood and generate electric current. The test unit is arranged on the portable housing and electrically connected with the lancing unit, and is used for detecting the intensity of the generated current and communicating with the outside. The lancet is arranged in the cavity of the portable housing.

The device further includes a lancet protection unit. The lancet is connected to the portable housing through a spring, and the lancet protection unit is embedded in the cavity of the portable housing, and is set at the front end of the lancet.

The lancet protection unit further comprises a protective head and a protective sleeve. The front end of the protective sleeve is provided with through-hole and the rear end of the protective sleeve is embedded in the cavity. The lancet can extend through the through-hole and the protection head is used to block the through-hole.

Furthermore, there are a plurality of lancing units. The amount is between 1 and 4.

Furthermore, these lancing units are provided with intervals between two, and are provided along the cavity opening of the portable housing.

Furthermore, the lancing unit is also covered with a protective film, which is equipped with a siphon groove. And the opening of the siphon groove is located at the cavity opening of the portable housing and communicated with the outside.

The test unit further includes a communication module and a current detection module, and the communication module is electrically connected to the current detection module.

Furthermore, the communication module is a near field communication chip.

The device further includes a temperature detection unit, which is equipped on the portable housing, and is electrically connected with the communication module.

The application also provides a blood glucose detection system, wherein the blood glucose detection system includes the blood glucose detection device of the aforementioned one aspect or the blood glucose detection device and a mobile terminal of another aspect. The mobile terminal is equipped to receive the current intensity sent by the blood glucose detection device and determine the blood glucose concentration according to the current intensity.

The mobile terminal, further comprising: a near field communication reading device, which is used to obtain the current intensity sent by the blood glucose detection device.

The blood glucose detection device further comprises a temperature detection unit and the mobile terminal is also used for receiving the temperature that the temperature detection unit obtains. If the temperature is in preset temperature range, then it determines the blood glucose concentration according to the current intensity.

Compared with the prior art, the application presents a blood glucose detection device and system, which can provide a portable blood glucose detection device for patients. The blood glucose detection device may include a plurality of lancing units, which collect patient blood and generate current. The current is detected by the test unit and can transmit the current intensity data to the external device, thus making the glucose detection device portable. Besides, there is no need to change test strips every time one performs a blood glucose test as the current intensity data can be transmitted externally for management, which improves the patient's experience and makes it convenient for patients to perform blood glucose tests when they go out.

### BRIEF DESCRIPTION OF THE DRAWINGS

Other features, objects and advantages of the present application will become more apparent with the detailed description of the non-limiting embodiments made with reference to the following drawings:

FIG. 1 is the structural diagram of the embodiment of the blood glucose detection device;

FIG. 2 is the siphon groove structural diagram of the embodiment of the blood glucose detection device;

FIG. 3 is the circuit structural diagram of the embodiment of the blood glucose detection device;

FIG. 4 is the structural diagram of the embodiment of the preferred blood glucose detection device;

FIG. 5 is the structural diagram of the other embodiment of the blood glucose detection device;

FIG. 6 is the structural diagram of the other embodiment of the blood glucose detection device;

FIG. 7 is the siphon groove structural diagram of the other embodiment of the blood glucose detection device;

FIG. 8 is the circuit structural diagram of the other embodiment of the blood glucose detection device.

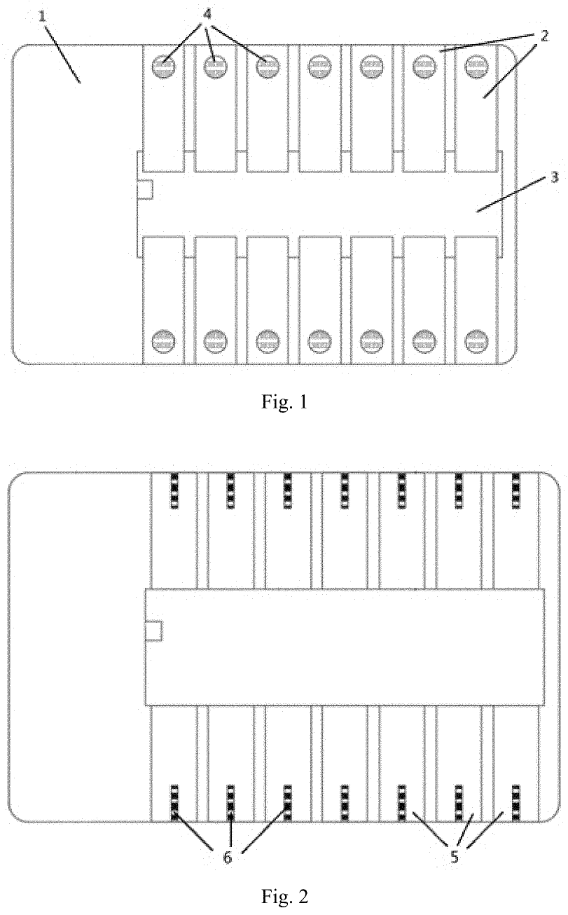

Description of the drawing elements: 1. portable card-type base; 2. lancing unit; 3. test unit; 4. groove; 5. protective film; 6. siphon groove; 7. communication module; 8. current detection module; 9. temperature detection unit; 10. portable housing; 11. lancet; 12. protective head; 13. protective sleeve.

The same or similar reference signs in the drawings represent the same or similar elements.

### DETAILED DESCRIPTION

The application will be described in further detail below in conjunction with the accompanying drawings.

In a typical configuration of the application, the terminal and the network device each include one or more processors (CPU), an input/output interface, a network interface and a memory.

The memory may include non-persistent memory, random access memory (RAM) and/or non-volatile memory in computer readable media, such as, read only memory (ROM) or flash memory (flash RAM). Memory is an example of a computer-readable medium.

Computer-readable media, including persistent and non-persistent, removable and non-removable media, can achieve information storage by any method or technology. The information may be computer readable instructions, data structures, modules of programs, or other data. Examples of computer storage media include, but are not limited to, phase-change random access memory (PRAM), static random access memory (SRAM), dynamic random access memory (DRAM), other types of random access memory (RAM), read only memory (ROM), electrically erasable programmable read only memory (EEPROM), flash memory or other memory technology, compact disc read only memory (CD-ROM), digital versatile disc (DVD) or other optical storage, magnetic tape cartridges, magnetic tape disk storage or other magnetic storage devices or any other non-transmission medium, that can be used to store information that can be accessed by a computing device. Computer-readable media, as defined herein, excludes non-transitory computer-readable media, such as modulated data signals and carrier waves.

The application provides a blood glucose detection device, wherein, the blood glucose detection device comprises a portable card-type base 1, at least one lancing unit 2 and a test unit 3. At least one lancing unit 2 is arranged on the edge of the portable card-type base 1. The lancing unit 2 includes a groove 4 provided on the portable card-type base 1. The groove 4 is coated with glucose oxidase or glucose dehydrogenase and the lancing unit 2 is used to collect blood and generate electric current. The test unit 3 is arranged on the portable card-type base 1 and electrically connected with the lancing unit 2, and is used for detecting the intensity of the generated current and communicating with the outside.

Wherein, the size and weight of the portable card-type base 1 are similar to bank cards, transportation cards, etc. commonly used in daily life, which are convenient for patients to carry when they go out, and can be placed in similar wallets and carry-on bags.

The lancing unit 2 is arranged on the portable card-type base 1, one end of which is arranged at the edge of the portable card-type base 1, and the other end is arranged in the middle area of the portable card-type base 1, and is used for collecting the blood of the patient and generating electricity according to the chemical reactions of blood flow. In order to facilitate the collection of the patient's blood, the embodiment of the application is also provided with a groove 4 for collecting blood on the portable card-type base 1. The groove 4 is set near the edge of the portable card base 1, and the inner wall of the groove 4 is also coated with glucose oxidase or glucose dehydrogenase for reacting with glucose in blood to generate electric current. The blood of the patient can be fully collected by the groove 4 to avoid blood dispersion, so that the blood can fully react with the glucose oxidase or glucose dehydrogenase in the groove 4 to avoid the error of blood glucose detection caused by insufficient reaction and improve the accuracy of blood glucose detection.

In the embodiment of the application, in order to reduce the trivial operation that the patient needs to replace the test strip when performing blood glucose detection each time, a plurality of disposable lancing units 2 are provided on the portable card-type base 1, and the specific number of lancing units 2 can be set according to the size and convenience of use of the portable card-based base 1. Preferably, the number can be set between 2 and 20.

In addition, in order to avoid blood contamination between the lancing units 2, a certain interval is set between each two lancing units 2, and the specific interval distance can be determined according to actual use requirements. The lancing units 2 can be arranged in sequence along the edge of the portable card-type base 1, and two groups of lancing units 2 can be simultaneously arranged on the two longer sides of the portable card-type base 1.

In the embodiment of the application, the lancing unit 2 is also covered with a protective film 5. As shown in FIG. 2, the protective film 5 can be used to protect the glucose oxidase or glucose dehydrogenase in the lancing unit from being subjected to external pollution or influence, thereby extending the service life and improving the accuracy of test. In addition, the protective film 5 is also provided with a siphon groove 6. The opening of the siphon groove 6 is at the edge of the portable card-type base 1 and communicates with the outside. The other end of the siphon groove 6 extends to the groove 4 in the lancing unit 2. The siphon groove 6 has a siphoning action that siphons the patient's blood located in the opening to the groove 4, thus eliminating the need for the patient to drip blood into the groove 4, facilitating the patient's blood collection operation while avoiding possible contamination near the groove 4 when dripping blood.

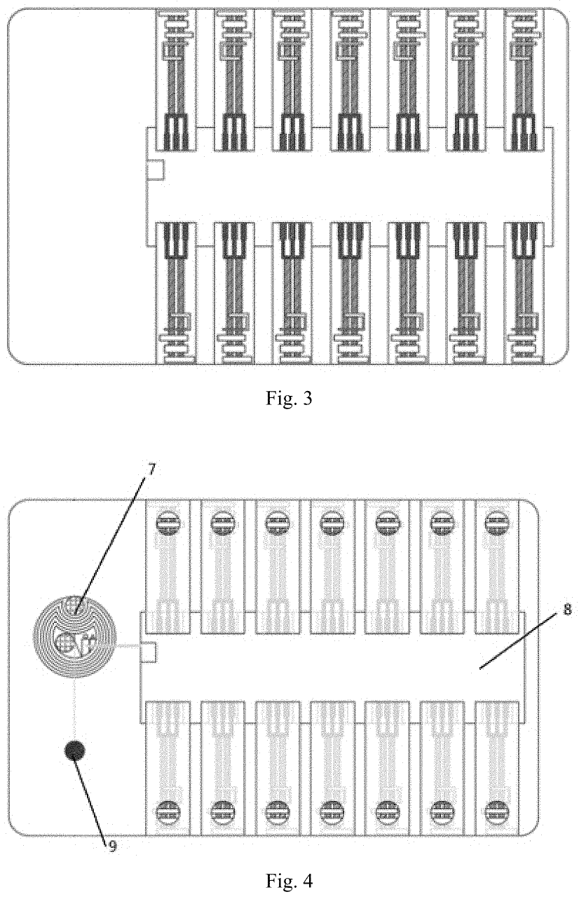

In addition, the lancing unit 2 is also electrically connected with the test unit 3 provided on the portable card-type base 1, and the lancing unit 2 transmits the generated current to the test unit 3. Specifically, the groove 4 in the lancing unit 2 is electrically connected to the test unit 3, and the current generated by the groove 4 is transmitted to the test unit 3 for current intensity detection through the current. The test unit 3 can transmit the current intensity data to an external device in real time, or can temporarily store the current intensity data and delay the transmission, or provide the corresponding current intensity data according to the data acquisition request of the external device.

Wherein, the connection circuit between the lancing unit 2 and the test unit 3 adopts the three-electrode system in electrochemical analysis, as shown in FIG. 3. Compared with the traditional two-electrode system, the three-electrode system includes working electrode, reference electrode and counter electrode. The reference electrode is used to set the zero point. The current flows through the working electrode and the reference electrode to form a system that is not or essentially less energized. The stability of the potential of the reference electrode is used to measure the electrode potential of the working electrode. The working electrode and the auxiliary electrode form an energized system that is used to measure the current flowing through the working electrode. The three-electrode measurement system is used to study the relationship between the point position of the working electrode and the current.

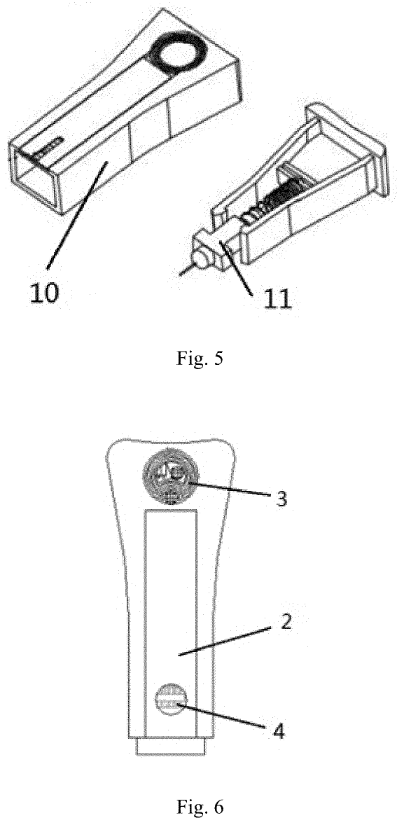

In the embodiment of the application, the test unit 3 may include a communication module 7 and a current detection module 8. As shown in FIG. 4, the communication module 7 is electrically connected to the current detection module 8. In addition, the lancing unit 2 is electrically connected to the current detection module 8, and the current generated by the lancing unit 2 is input to the current detection module 8 for current intensity detection. Preferably, the current detection module 8 may be a current detection chip, which receives the current signal output by the lancing unit 2 through the two electrodes via the three electrodes.

Preferably, the communication module 7 is a near-field communication chip, and near-field communication (NFC) is a short-distance high-frequency wireless communication technology. “Near field” refers to the proximity of electromagnetic field radio waves, which can realize exchange data in close proximity to each other. It evolved from the integration of contactless radio frequency identification (RFID) and interconnection and interoperability technology, which can integrate the functions of inductive reader, inductive card and peer-to-peer communication on a single chip.

In the embodiment of the present application, the lancing unit also includes a temperature detection unit 9, which is equipped on the portable card-type base 1, and is electrically connected with the communication module 7. Wherein, since blood glucose needs to be tested within a certain temperature range to achieve the highest accuracy. For example, the most accurate test is between 18 degrees Celsius and 25 degrees Celsius. Therefore, the temperature during blood sugar detection is obtained through the temperature detection unit 9, which can be used for judging whether the obtained blood glucose test results are meaningful.

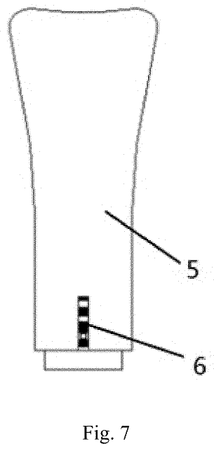

The other embodiment of the application also provide a blood glucose detection device. As shown in FIGS. 5 and 6, the blood glucose detection device comprises a portable housing 10 with cavity, lancet 11, at least one lancing unit 2 and a test unit 3. At least one lancing unit 2 is arranged on the cavity opening side of the portable housing 10. The lancing unit 2 includes a groove 4 provided on the portable housing 10. The groove 4 is coated with glucose oxidase or glucose dehydrogenase and the lancing unit 2 is used to collect blood and generate electric current. The test unit 3 is arranged on the portable housing 10 and electrically connected with the lancing unit 2, and is used for detecting the intensity of the generated current and communicating with the outside. The lancet 11 is arranged in the cavity of the portable housing 10.

Wherein, the lancet 11 is integrated into the blood glucose detection device, realizing the integration of the patient's blood acquisition and blood test, which is convenient for the patient to use, and avoids the inconvenience caused by the separation of the lancet and the blood glucose detection device.

The lancing unit 2 is arranged on the portable housing 10, one end of which is arranged at one side of the cavity opening of portable housing 10, and the other end is arranged at one side away from the cavity opening of the portable housing 10, and is used for collecting the blood of the patient and generating electricity according to the chemical reactions of blood flow. In order to facilitate the collection of the patient's blood, some embodiments of the application are also provided with a groove 4 for collecting blood on the portable housing 10. The groove 4 is set near the edge of the portable housing 10, and the inner wall of the groove 4 is also coated with glucose oxidase or glucose dehydrogenase for reacting with glucose in blood to generate electric current. The blood of the patient can be fully collected by the groove 4 to avoid blood dispersion, so that the blood can fully react with the glucose oxidase or glucose dehydrogenase in the groove 4 to avoid the error of blood glucose detection caused by insufficient reaction and improve the accuracy of blood glucose detection.

Preferably, in order to avoid the lancet 11 accidentally injuring the patient when blood collection is not required, the device also includes a lancet protection unit. Wherein, the lancet 11 is connected to the portable housing 10 through a spring, and the lancet protection unit is embedded in the cavity of the portable housing 10, and is set at the front end of the lancet 11.

Specifically, the lancet protection unit includes a protective head 12 and a protective sleeve 13. The front end of the protective sleeve 13 is provided with a through-hole while the rear end is embedded in the cavity of the portable housing 10. And the lancet 11 can extend through the through-hole. One end of the protective head 12 is provided with a protrusion, which is made of soft material and can be used to block the through-hole at the front of the protective sleeve 13. And lancet 11 can be pierced into the protrusion to prevent the front tip of lancet 11 from stabbing the patient.

In the embodiment of the application, in order to reduce the trivial operation that the patient needs to replace the test strip when performing blood glucose detection each time, a plurality of disposable lancing units 2 are provided on the outer wall of portable housing 10, and the specific number of lancing units 2 can be set according to the size and convenience of use of the portable housing 10. Preferably, the number can be set between 1 and 4.

In addition, in order to avoid blood contamination between the lancing units 2, a certain interval is set between each two lancing units 2, and the specific interval distance can be determined according to actual use requirements. The lancing units 2 can be arranged around the cavity opening of the portable housing 10.

In the embodiment of the application, the lancing unit 2 is also covered with a protective film 5. As shown in FIG. 7, the protective film 5 can be used to protect the glucose oxidase or glucose dehydrogenase in the lancing unit from being subjected to external pollution or influence, thereby extending the service life and improving the accuracy of test. In addition, the protective film 5 is also provided with a siphon groove 6. The opening of the siphon groove 6 is at the cavity opening of the portable housing 10 and communicates with the outside. The other end of the siphon groove 6 extends to the groove 4 in the lancing unit 2. The siphon groove 6 has a siphoning action that siphons the patient's blood located in the opening to the groove 4, thus eliminating the need for the patient to drip blood into the groove 4, facilitating the patient's blood collection operation while avoiding possible contamination near the groove 4 when dripping blood.

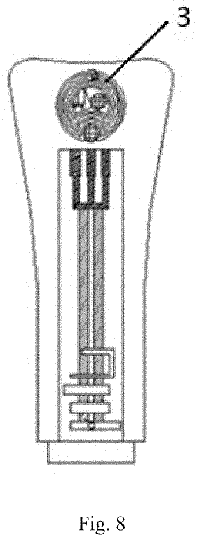

In addition, the lancing unit 2 is also connected with the test unit 3 provided on the portable housing 10 through a circuit, and the lancing unit 2 transmits the generated current to the test unit 3. Specifically, the groove 4 in the lancing unit 2 is connected to the test unit 3 through a circuit, and the current generated by the groove 4 is transmitted to the test unit 3 for current intensity detection through the current. The test unit 3 can transmit the current intensity data to an external device in real time, or can temporarily store the current intensity data and delay the transmission, or provide the corresponding current intensity data according to the data acquisition request of the external device.

Wherein, the connection circuit between the lancing unit 2 and the test unit 3 adopts the three-electrode system in electrochemical analysis, as shown in FIG. 8.

In the embodiment of the application, the test unit 3 may include a communication module 7 and a current detection module 8 (not shown in the figure). The communication module 7 is electrically connected to the current detection module 8. In addition, the lancing unit 2 is electrically connected to the current detection module 8, and the current generated by the lancing unit 2 is input to the current detection module 8 for current intensity detection. Preferably, the current detection module 8 may be a current detection chip, which receives the current signal output by the lancing unit 2 through the two electrodes via the three electrodes.

Preferably, the communication module 7 is a near field communication chip.

In some embodiments of the present application, the lancing unit also includes a temperature detection unit 9 (not shown in the figure), which is equipped on the portable housing 10, and is electrically connected with the communication module 7.

According to another aspect of the application, the application also provides a blood glucose detection system. The blood glucose detection system includes any one of the aforementioned two blood glucose detection devices and a mobile terminal. The mobile terminal is equipped to receive the current intensity sent by the blood glucose detection devices and determine the blood glucose concentration according to the current intensity.

Specifically, the mobile terminal includes a near-field communication reading device, which can be used to obtain the current intensity sent by the glucose detection device. After the mobile terminal obtains the current intensity data, it then calculates according to the formula corresponding to the current intensity and glucose concentration to obtain the corresponding glucose concentration data, i.e., blood glucose data. Preferably, a corresponding software application can be installed on the mobile terminal to calculate the blood glucose data.

In the embodiment of the application, the blood glucose detection device also comprises a temperature detection unit and the mobile terminal is also used for receiving the temperature that the temperature detection unit obtains. If the temperature is in preset temperature range, then it determines the blood glucose concentration according to the current intensity. Wherein, the mobile terminal can determine whether the obtained current intensity data is meaningful according to the obtained temperature. If it is within the preset temperature range, then the test is considered normal and the obtained current intensity data is valid. Then the current intensity data can be used to determine the blood glucose. Otherwise, the test is considered abnormal, and the obtained current intensity data is a problematic data. At this time, the mobile terminal can remind the patient that there is a problem with the blood glucose test, and a re-test may be needed.

In conclusion, the application provides presents a blood glucose detection device and system, which can provide a portable blood glucose detection device for patients. The blood glucose detection device may include a plurality of lancing units, which collect patient blood and generate current. The current is detected by the test unit and can transmit the current intensity data to the external device, thus making the glucose detection device portable. Besides, there is no need to change test strips every time one performs a blood glucose test as the current intensity data can be transmitted externally for management, which improves the patient's experience and makes it convenient for patients to perform blood glucose tests when they go out.

It should be noted that the application may be implemented in software and/or a combination of software and hardware, such as an application specific integrated circuit (ASIC), a general-purpose computer, or any other similar hardware device. In one embodiment, the software program of the application may be executed by a processor to implement the steps or functions described above. Likewise, the software programs of the application (including associated data structures) may be stored on a computer-readable recording medium, such as an RAM memory, magnetic or optical drives or floppy disks, and the like. In addition, some steps or functions of the application may be implemented in hardware. For example, a circuit may cooperate with a processor to perform various steps or functions.

In addition, a part of the application can be applied as a computer program product. Take a computer program instruction as an example. When it is executed by a computer, the method and/or technical solution according to the application can be invoked or provided by the operation of that computer. The program instructions that invoke the methods of the application may be stored in fixed or removable recording media, and/or transmitted via data streams in broadcast or other signal-bearing media, and/or stored in the working memory of the computer device on which the program instructions are executed. Wherein, an embodiment according to the application includes an apparatus. The apparatus includes a memory for storing computer program instructions and a processor for executing the program instructions, wherein: when the computer program instructions are executed by the processor, the apparatus is triggered to run the method and/or technical solution based on the preceding multiple embodiments according to the application.

For those skilled in the technology, it is obvious that the application is not limited to the details of the above-mentioned exemplary embodiments, and that the application can be implemented in other specific forms without departing from the spirit or essential characteristics of the application. Accordingly, the embodiments are to be regarded not as restrictive but as illustrative in all respects. The scope of this application is limited by the appended claims, rather than by the above description, and is therefore intended to encompass all variations that fall within the meaning and scope of the equivalent elements of the claims. Any attached drawings in the claims shall not be construed as limiting the involved claim. Furthermore, it is clear that the word “comprising” does not exclude other units or steps and the singular does not exclude the plural. A plurality of units or means recited in the device claims can also be realized by one unit or means by means of software or hardware. The terms first, second, etc. are used to denote names instead of any particular order.

1. A blood glucose detection device, comprising:
a portable card-type base,
at least one lancing unit, and
a test unit,
wherein the at least one lancing unit is arranged on the edge of the portable card-type base, and the lancing unit includes a groove provided on the portable card-type base, the groove is coated with glucose oxidase or glucose dehydrogenase and the lancing unit is used to collect blood and generate electric current, and the test unit is arranged on the portable card-type base and electrically connected with the lancing unit, and is used for detecting the intensity of the generated current and communicating with the outside.

2. The blood glucose detection device of claim 1, wherein the lancing devices are plurality, the amount is between 2 and 20.

3. The blood glucose detection device of claim 2, wherein these lancing units are provided with intervals between two, and are provided along the edge of the portable card-type base.

4. The blood glucose detection device of claim 1, wherein the lancing unit is covered with a protective film, which is equipped with a siphon groove, and the opening of the siphon groove is located at the edge of the portable card-type base and communicated with the outside.

5. The blood glucose detection device of claim 1, wherein the test unit comprises a communication module and a current detection module, and the communication module is electrically connected to the current detection module.

6. The blood glucose detection device of claim 5, wherein the communication module is a near field communication chip.

7. The blood glucose detection device of claim 5, wherein the device further comprises a temperature detection unit, which is equipped on the portable card-type base, and is electrically connected with the communication module.

8. A blood glucose detection device, comprising:
a portable housing with cavity,
a lancet,
at least one lancing unit, and
a test unit,
wherein the at least one lancing unit is arranged on the cavity opening side of the portable housing, and the lancing unit includes a groove provided on the portable housing, the groove is coated with glucose oxidase or glucose dehydrogenase and the at least one lancing unit is used to collect blood and generate electric current, and the test unit is arranged on the portable housing and electrically connected with the lancing unit, and is used for detecting the intensity of the generated current and communicating with the outside, the lancet is arranged in the cavity of the portable housing.

9. The blood glucose detection device of claim 8, further comprising: a lancet protection unit, the lancet is connected to the portable housing through a spring, and the lancet protection unit is embedded in the cavity of the portable housing, and is set at the front end of the lancet.

10. The blood glucose detection device of claim 9, wherein the lancet protection unit comprises a protective head and a protective sleeve, the front end of the protective sleeve is provided with through-hole and the rear end of the protective sleeve is embedded in the cavity, the lancet can extend through the through-hole and the protection head is used to block the through-hole.

11. The blood glucose detection device of claim 8, wherein the lancing units are plurality, the amount is between 1 and 4.

12. The blood glucose detection device of claim 9, wherein these lancing units are provided with intervals between two, and are provided along the cavity opening of the portable housing.

13. The blood glucose detection device of claim 8, wherein the lancing unit is covered with a protective film, which is equipped with a siphon groove, and the opening of the siphon groove is located at the cavity opening of the portable housing and communicated with the outside.

14. The blood glucose detection device of claim 8, wherein the test unit comprises a communication module and a current detection module, and the communication module is electrically connected to the current detection module.

15. The blood glucose detection device of claim 14, wherein the communication module is a near field communication chip.

16. The blood glucose detection device of claim 14, wherein the device further comprises a temperature detection unit, which is equipped on the portable housing, and is electrically connected with the communication module.

17. A blood glucose detection system comprising the blood glucose detection device according to claim 1 and a mobile terminal, wherein the mobile terminal is equipped to receive the current intensity sent by the blood glucose detection device and determine the blood glucose concentration according to the current intensity.

18. The blood glucose detection system of claim 17, wherein the mobile terminal comprises a near field communication reading device, which is used to obtain the current intensity sent by the blood glucose detection device.

19. The blood glucose detection system of claim 17, wherein the blood glucose detection device further comprises a temperature detection unit and the mobile terminal is used for receiving the temperature that the temperature detection unit obtains, if the temperature is in preset temperature range, it determines the blood glucose concentration according to the current intensity.

20. A blood glucose detection system comprising the blood glucose detection device according to claim 8 and a mobile terminal, wherein the mobile terminal is equipped to receive the current intensity sent by the blood glucose detection device and determine the blood glucose concentration according to the current intensity.

21. The blood glucose detection system of claim 20, wherein the mobile terminal comprises a near field communication reading device, which is used to obtain the current intensity sent by the blood glucose detection device.

22. The blood glucose detection system of claim 20, wherein the blood glucose detection device further comprises a temperature detection unit and the mobile terminal is used for receiving the temperature that the temperature detection unit obtains, if the temperature is in preset temperature range, it determines the blood glucose concentration according to the current intensity.
